(19) United States
(12) Patent Application Publication
(10) Pub. No.: US 2007/0288080 A1
(43) Pub. Date: Dec. 13, 2007

(54) Stent expanding device

(76) Inventors: Michael W. Maccollum, Poway, CA (US); Michael R. Bialas, San Diego, CA (US); David A. Mackiewicz, Scotts Valley, CA (US)

Correspondence Address: FULWIDER PATTON LLP, HOWARD HUGHES CENTER, 6060 CENTER DRIVE, TENTH FLOOR, LOS ANGELES, CA, 90045, US

(21) Appl. No.: 11/449,451

(22) Filed: Jun. 7, 2006

(51) Int. Cl. A61F 2/06

(52) U.S. Cl. 623/1.11

(57) ABSTRACT

side and a pair of pins for insertion into the sleeve. In another embodiment, the device has an expanding mandrel having a side, two open ends and a plurality of longitudinal slots along the side. An expansion pin is included, and has a cylindrical body with a tapered tip. In another embodiment, the device expands a stent into a tapered stent. The tapered stent expanding device includes a slotted expansion mandrel and a tapered pin having an expansion crown. In another aspect, the device includes a threaded mandrel for the uniform expansion of the diameter of a stent.

### BACKGROUND OF THE INVENTION

The invention relates generally to minimum stress expansion devices for stents that limit the stresses applied to stent struts in the expansion process of stents.

The term stent generally refers to a prosthesis, which can be introduced into a corporeal lumen and expanded to support that lumen or attach a conduit to the inner surface of that lumen. Stents made of shape settable material are generally known in the art. Stents are generally either balloon expandable or self-expanding devices. A balloon expandable stent is delivered within vasculature mounted on a balloon catheter and expanded at an interventional site to accomplish implantation. The self-expanding stent is compressed into a reduced size having an outer diameter substantially smaller than the stent in its expanded shape. The stent is held in its compressed state during its passage through the patient's vascular system until reaching the target treatment site, whereupon the compressed self-expanding stent may be deployed. While in its compressed state, stress is stored in the bends of the stent limbs. During deployment, the stresses in the stent limbs cause the stent to expand radially from its initially compressed state. Once in place, the radial extremities of the stent bear against the inside walls of the passageway, thereby allowing normal blood flow.

One particular type class of shape settable materials that are practical for stents include Nickel-Titanium alloys (Nitinol). Previous methods to set a desired expanded Nitinol stent configuration involved forcing the stent over a cylindrical mandrel matching the desired inner diameter of the stent. The stent is then heat treated until the shape memory of the stent in its austenite phase has a diameter matching that of the mandrel. This process results in producing a stent that does not store stress in an optimal manner. Certain current approaches to stent expansion processes utilize the superelastic properties of Nitinol by creating a phase transformation in the stent as its diameter is enlarged. Alternatively, the stent may be tapered and, for example, transition from a seven millimeter (mm) diameter to a ten mm diameter over a thirty mm or forty mm length. Likewise, as with the uniform diameter stent, previous methods employed to expand a stent into a tapered stent involved forcing the stent onto a tapered mandrel and heating. Such methods suffer similar drawbacks to the methods used to uniformly expand stents.

During the expansion process, the mechanical stress in the Nitinol causes a phase transformation from austenite to martensite to accomplish a change in diameter. Stents may be chilled to lower temperatures to transform them to martensite as a way to lower the forces required by an operator to perform the expansion process. Once the stent has been shaped to the increased diameter, a heat treatment process at approximately 525 degrees Centigrade (° C.) is used to transform the atomic structure of the stent back to austenite and relieve built up internal stresses.

Current expansion tooling consists of a cylindrical mandrel with a tapered end. In order to perform shape setting, an operator may use a push-pull technique to load the stent over the tapered mandrel. The superelastic property of Nitinol allows it to recover from up to eight to ten percent strain without deformation. The theoretical plane strain of a stent strut is up to six percent for practical expansions steps, when considering ideal radial expansion only. Additional strain provided by this technique may result in an amount of strain which exceeds the capability of the material to recover without deformation. Such a result is also associated with stents formed from materials which are not inherently superelastic. Inspection is required to determine whether further processing is needed to overcome the effects of this deformation. The repetitive nature of the push-pull technique can also lead to carpel tunnel syndrome in the operator.

Previous methods employed successive one to two millimeter expansions of stents by employing mandrels of successively larger diameters. Though so intended, these methods did not eliminate the presence of cracks and notch defects. Notch defects occur after the post expansion treatment of a cracked stent.

An improved method is needed, therefore, which accomplishes diameter expansion of an expandable or self-expanding stent by applying forces to the stent in an outward radial direction only. Such a technique would limit the strain of individual struts to a minimal level while eliminating the presence of longitudinal forces on the stent during the process of loading the stent onto shape setting tooling. The elimination of longitudinal forces is especially important for stents of designs that do not have sufficient longitudinal rigidity, as such designs are especially susceptible to excessive elongation or contraction. An improved method is desired that will minimize the potential for injury to the operator, that is also capable of automation and reduces or eliminates the influence of human error.

The present invention satisfies these and other needs.

### SUMMARY OF THE INVENTION

Briefly and in general terms, the present invention is directed towards a stent expanding device for expanding the dimensions of stents.

In one embodiment, the stent expanding device may uniformly expand the diameter of a stent. The device includes an expansion mandrel having a side, two open ends and a plurality of longitudinal slots along the mandrel's side. The device also includes an expansion pin having a cylindrical body and a tapered end. The expansion pin has an upper cylindrical body having a side face, a top face, and may include a closed channel in the side face and an open channel in the top face.

In one aspect, the stent expanding device may include an expanding mandrel that has a cavity and a cut along the length of the side. The device also includes an expansion pin having a tapered tip. The mandrel may include overlapping members configured to fit into receiving grooves in the mandrel. In a method of use, a stent is loaded onto a mandrel that has a cavity and a cut along the length of the side. An expansion pin is then inserted into the cavity of the mandrel.

An alternative embodiment of the stent expanding device has an expansion mandrel having a cavity, a side, two open ends, and a plurality of longitudinal slots along the side. The slots are spaced at regular intervals and have a terminus that is continuous with the ends of the mandrel. The slots commence at a keyhole in the side of the mandrel, and each consecutive slot terminates at an end of the mandrel opposite to the end of the preceding slot. The device has an expansion pin having a cylindrical body and a tapered end, an expansion crown that has teeth and a bore. The teeth are configured to slide into the termini of the slots and the bore is configured to receive the cylindrical body of the expansion pin. A rounding rod is provided and is configured to be received into the cavity of the mandrel. In a method of use, an expansion pin is inserted into an expansion crown to form a loaded expansion pin. A stent is slid onto a mandrel to form a loaded mandrel. The loaded expansion pin is then partially inserted into each end of the mandrel. Expansion crowns are slid down the expansion pins until fingers of the crowns are seated in slots of the mandrel. The loaded expansion pin is completely inserted into each end of the mandrel to form an expanded stent.

Another embodiment of the stent expanding device expands the stent into a tapered stent. The device includes a tapered mandrel that has a closed end, an open end, and slots along the side of the tapered mandrel. The slots extend from the closed end of the mandrel and terminate at and are continuous with the open end of the mandrel. The device also includes a tapered expansion pin that has a tapered cylindrical body. The tapered stent expanding device may further include teeth on the tapered expansion pin. The teeth are configured to be received into the termini of the slots. The tapered stent expanding device may further include a contraction ring that has a circular passageway. The passageway is configured to receive the tapered mandrel. In another method of use, a contraction ring is slid over a mandrel that has a closed end and an open end. A stent is loaded onto the mandrel from the closed end. The contraction ring is slid away from the stent. A tapered pin is inserted into the open end of the mandrel.

In another aspect, the stent expanding device includes a threaded mandrel that has a tapered tip. The device further includes a rotating device for rotating the threaded mandrel. In a method of use, a threaded mandrel that has a tapered tip is rotated, and a stent is fed onto the mandrel while the mandrel rotates.

The above described device has broad applicability to stents made of any shape settable material. Other features and advantages of the present invention will become apparent from the following detailed description, taken in conjunction with the accompanying drawings, which illustrate, by way of example, the principles of the invention.

### BRIEF DESCRIPTION OF THE DRAWINGS

FIG. 1 is a perspective view, depicting a stent expanding device of the present matter;

FIG. 2 is a perspective view, depicting another embodiment of an expanding mandrel of the present invention, in an expanded state;

FIG. 3 is a perspective view, depicting the expanding mandrel of FIG. 2, in an unexpanded state;

FIG. 4 is a perspective view, depicting another embodiment of a stent expanding device in an expansion kit;

FIG. 5 is a perspective view, depicting an expansion pin;

FIG. 6 is a perspective view, depicting a rounding rod;

FIG. 7 is a perspective view, depicting an expansion crown;

FIG. 8 is a perspective view, depicting the expansion crown of FIG. 7 loaded on the expansion pin of FIG. 5;

FIG. 9 is a perspective view, depicting the expanding mandrel of FIG. 4;

FIG. 10 is a perspective view, depicting the expanding mandrel of FIG. 9 with the rounding rod of FIG. 6 inserted therein;

FIG. 11 is a perspective view, depicting the assembly of FIG. 10 having a stent loaded thereon;

FIG. 12 is an enlarged partial view, depicting the mandrel having a stent loaded thereon;

FIG. 13 is a perspective view, depicting the mandrel of FIG. 9 having two stents loaded thereon;

FIG. 14 is a perspective view, depicting the loaded expanding mandrel of FIG. 13 in operation with a pair of loaded expansion pins;

FIG. 15 is an enlarged partial view, depicting the loaded expanding mandrel of FIG. 14 with a loaded expansion pin further advanced into the mandrel;

FIG. 16 is a perspective view, depicting a stent expanding device with the pins completely inserted and a pair of expanded stents loaded thereon;

FIG. 17 is a perspective view, depicting a tapered stent expanding device;

FIG. 18 is a perspective view, depicting a contraction ring in operation with the tapered stent expanding device of FIG. 17 without the tapered expansion pin;

FIG. 19 is a perspective view, depicting the tapered expanding mandrel of FIG. 18 with a stent loaded thereon;

FIG. 20 is a perspective view, depicting the tapered expansion pin of FIG. 17 in operation with the tapered expanding mandrel of FIG. 17;

FIG. 21 is an enlarged partial view, depicting a tapered expansion pin seated completely in the tapered expanding mandrel;

FIG. 22 is a plan view, depicting the tapered stent expanding device in combination with handling structures;

FIG. 23 is a cross-sectional view, depicting a terminal end portion of a tapered threaded mandrel;

FIG. 24 is a plan view, depicting the tapered threaded mandrel of FIG. 24 in operation with a rotating device; and

FIG. 25 is a cross-sectional view, depicting the tapered threaded mandrel of FIG. 24 with a stent loaded thereon.

### DETAILED DESCRIPTION OF THE PREFERRED EMBODIMENTS

Turning now to the figures, which are provided for example and not by way of limitation, there is shown the stent expanding device of the present invention. Incorporated into the stent expanding device are components designed to integrate with each other to expand a stent to an expanded diameter. The device is appropriate for both open cell and closed cell stents.

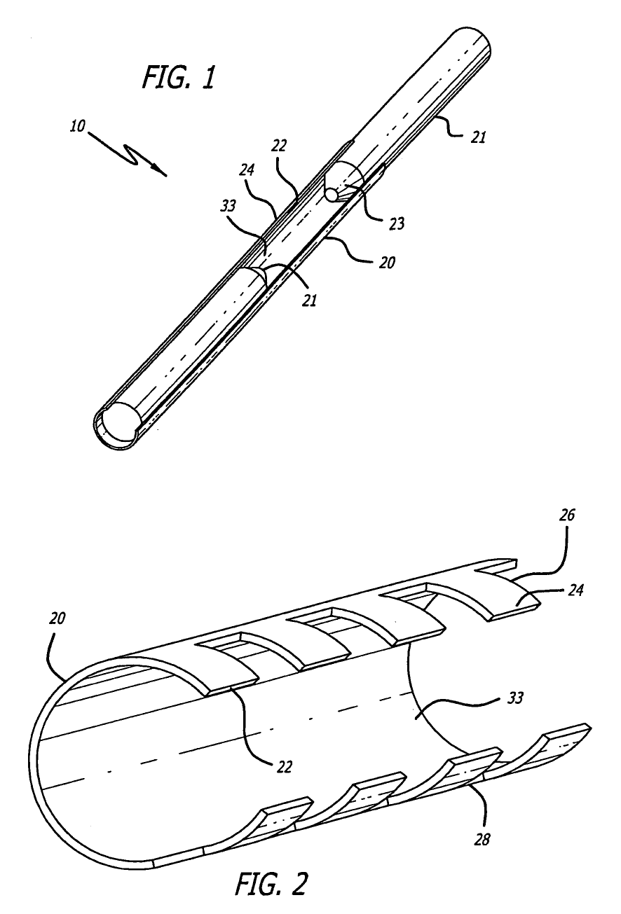

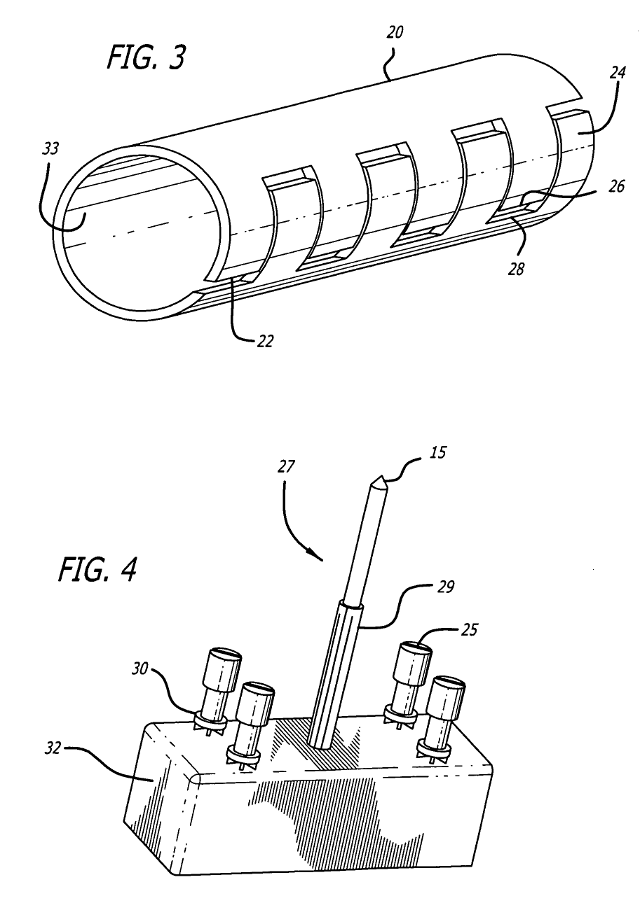

Referring to the drawings, which are provided for purposes of illustration and by way of example, the present invention provides for a stent expanding device 10 for stents and a method of using same. In one aspect, as shown in FIG. 1, the device 10 includes an expanding mandrel 20, and expansion pins 21. The pins 21 are generally in the shape of cylinder with a tapered end 23 for ease of insertion into the expanding mandrel 20. The expanding mandrel 20 is shaped in the form of a hollow right cylinder with a cut 22 along the length of a side 24 and includes a hollow cavity 33. In another aspect, as shown in FIGS. 2 and 3, the mandrel 20 has overlapping members 26 that fit into corresponding receiving grooves 28. In operation, a stent to be expanded is loaded entirely onto the mandrel 20 and the expansion pins 21 are inserted very close in time to one another, into the cavity 33 of the mandrel 20. The stent is expanded by the insertion of the pins 21. After undergoing a shape setting procedure and subsequent removal of the pins 21, the stent may be removed from the mandrel 20. The mandrel 20 can be biased to remain in an unexpanded state to facilitate removal of the expanded stent.

In another aspect, as shown in FIG. 4, a stent expanding device 27 includes a rounding rod 15, an expanding mandrel 29, expansion pins 25 and expansion crowns 30. The device may be provided in kit form, neatly stored in a base 32. The device 27 can be made of Inconel® or another suitable material that does not deform or shape set at a heat treatment temperature of 525° C.

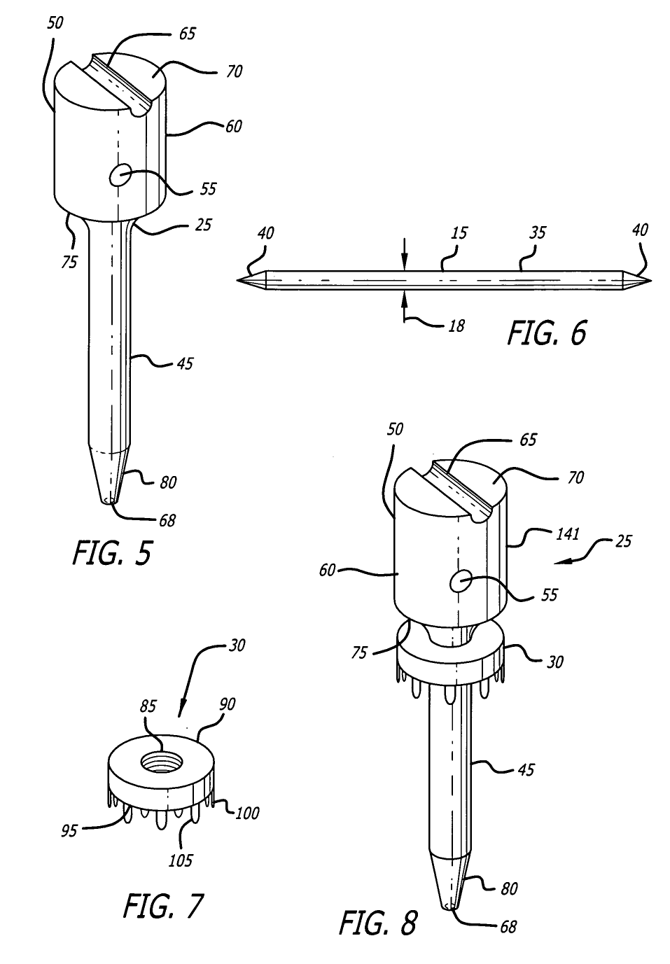

With reference to FIGS. 5 and 6, the rounding rod 15 has a cylindrical body 35 with two conical shaped tips 40. As shown in FIG. 5, the expansion pin 25 has a lower cylindrical body 45 connected to an upper cylindrical body 50. The upper cylindrical body 50 has a closed channel 55 in a side face 60 and an open channel 65 in a top face 70 of the upper cylindrical body 50. The lower cylindrical body 45 is connected to and coaxial with a bottom face 75 of the upper cylindrical body 50 and terminates at a tapered conical end 80. The closed channel 55 and open channel 65 can be used in the handling of the stent expanding device 27 as described in further detail below. The expansion pin 25 may have an internal bore 68 for receiving the rounding rod 15.

As shown in FIG. 7, the expansion crown 30 is generally shaped as a right cylinder with a bore 85 through a center of the structure. The crown 30 has a top face 90 and a bottom face 95. The bore 85 is sized and shaped to receive the lower cylindrical body 45 of the expansion pin 25, as shown in FIG. 8. Along the bottom face are fingers 100, symmetrically located every thirty degrees although other spacing including variable spacing is contemplated. The fingers 100 have tapered tips 105. In one aspect, the top face 90 of the crown 30 can be integral with the bottom face 75 of the pin 25.

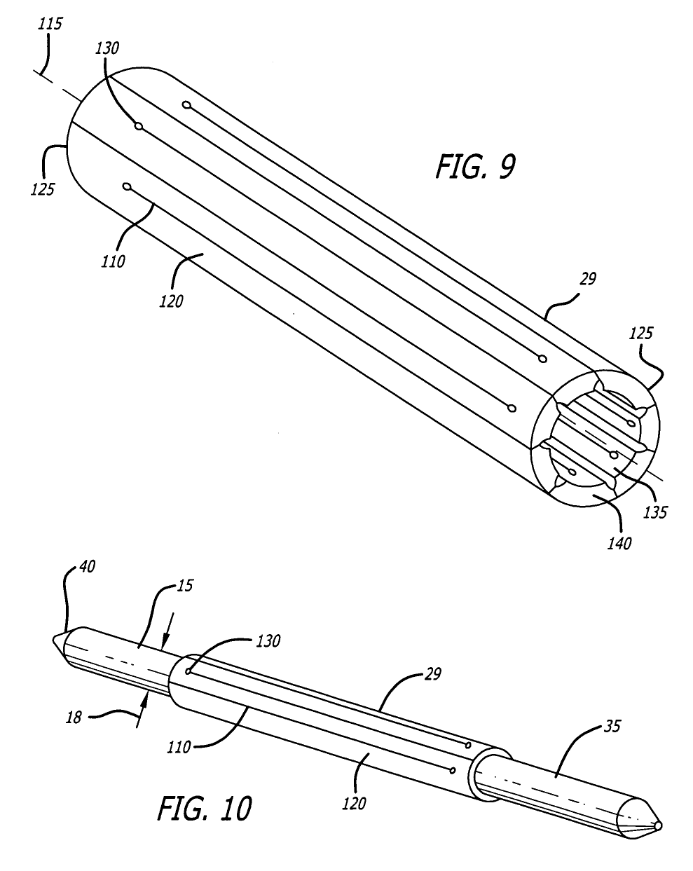

The expanding mandrel 29, as shown in FIG. 9, is generally in the shape of a right cylinder. The mandrel 29 has slots 110 that run parallel to a long axis 115 of the mandrel 29 and located symmetrically around a side 120 of the mandrel 29 at thirty degree intervals. It is to be recognized, however, that various numbers of slots can be incorporated into the mandrel 29 and accordingly, such slots can be spaced optimally about the mandrel 29. In one particular embodiment, the slots 110 commence at and are also open at the ends 125 of the mandrel 29 and run along a substantial length of the mandrel 29 and terminate at keyholes 130. The slots 110 alternate in orientation such that each slot 110 is located between two other slots 110 that commence at an opposite end 125 of the mandrel 29. The mandrel 29 has a hollow interior cavity 135 with a diameter approximately equal in size to a diameter 18 of the rounding rod 15. The mandrel 29 has a smooth transition portion 140 between the end 125 and the cavity 135 and in certain applications is curved inward for facilitating reception of the lower cylindrical body 45 of the expansion pin 25 into the cavity 135, as shown in FIG. 10.

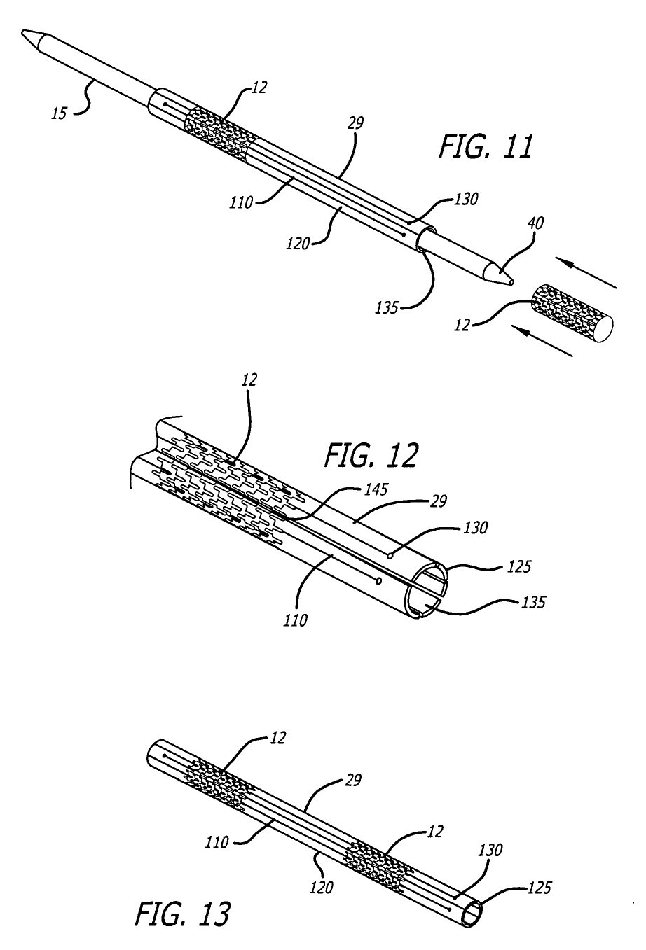

Referring now to FIGS. 11-16, a method of using the stent expanding device 27 is described. FIG. 11 depicts the rounding rod 15 inserted into the cavity 135 of an expanding mandrel 29 that is, for example, 2-6 millimeters in diameter to form the mandrel 29 into a smooth circular tube. As shown in FIGS. 11 and 12, a stent 12 is then slid onto mandrel 29 to form a loaded mandrel. If the stent 12 is 40 millimeters or less in length, for example, two stents 12 may be placed on the mandrel 29, as shown in FIG. 13. The stents 12 are placed completely between the keyholes 130, which are located near both ends 125 of the mandrel 29. The spines 145 of the stents 12 can be aligned with the slots 110. Next, the rounding rod is removed from the mandrel 29. The loaded mandrel is then immersed in alcohol to cool the stent 12 to approximately −10° C. The cooling causes a Nitinol stent to transition to the martensite phase in order to reduce the forces exerted by the operator in the loading of the shape setting tool. Accordingly, it is contemplated that not all stents require the same cooling step.

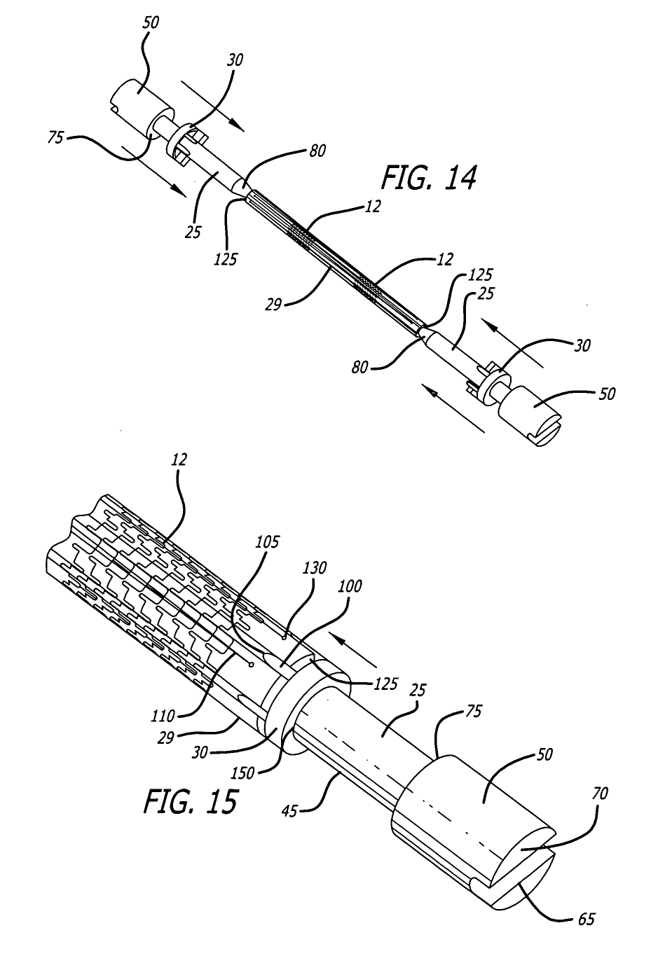

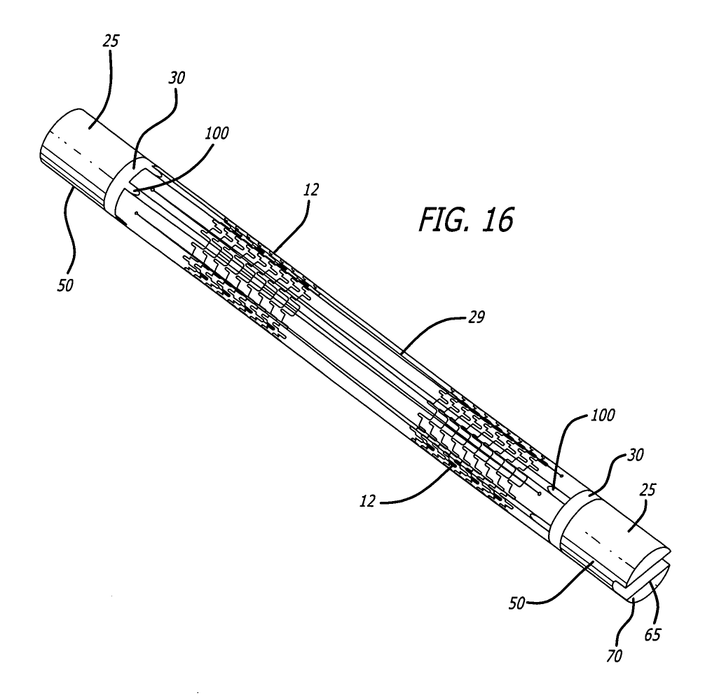

Thereafter, as shown in FIG. 14, the expansion pins 25 are inserted into the expansion crowns 30 to form loaded expansion pins (see FIG. 8). After cooling of the stents 12 is completed, the loaded mandrel 29 is removed from the alcohol and a loaded expansion pin 25 is inserted straight into each end 125 of the mandrel 29, to a midpoint 150 of the expansion pin 25, as shown in FIGS. 14 and 15. The rounding rod 15 may be received into the cavity 68, when the pins 25 are inserted into the mandrel 29 or the rounding rod 15 is removed prior to placing the expansion pins 25 into the loaded mandrel 29. As shown in FIG. 15, the expansion crowns 30 are slid down the expansion pins 25 until the fingers 100 of the crowns 30 are seated in the slots 110 of the mandrel 29. The pins 25 are then pressed completely into the mandrel 29, as shown in FIG. 16. With the aid of a microscope, if necessary, the spines 145 of the stent 12 are aligned with the slots 110. The stent 12 is then treated in a salt solution, and thereafter, the expanded stent 12 and the stent expanding device 27 are heat treated at a temperature of 525° C. The foregoing procedure may be repeated with successively larger mandrels 29 until the stent 12 is expanded to the desired size. The above described process may reversed to removed the stent 12 from the device 27. Final stent diameters may range from five mm to ten mm for uniform diameter stents.

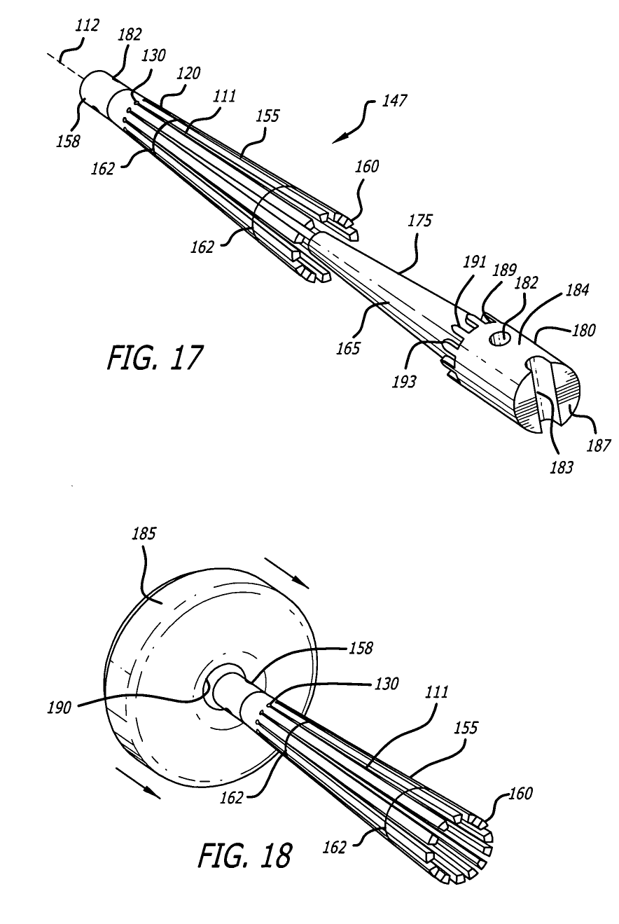

In another aspect, as shown in FIGS. 17-22, the stent expanding device 147 includes a tapered expanding mandrel 155. The tapered mandrel 155 has a closed end 158 and an open end 160. Referring to FIG. 17, the tapered mandrel 155 has slots 111 that run along a long axis 112 of the tapered mandrel 155 and located symmetrically around a side 120 of the tapered mandrel 155 at regular intervals (for example, between 15 and 40 degree intervals). The slots 111 commence at, and are also open at, the open end 160 of the tapered mandrel 155 and run along a substantial length of the tapered mandrel 155 and terminate at keyholes 130. Indicator marks 162 are provided on the side 120 to aid in the proper placement of the stent 12 on the mandrel 155.

As shown in FIGS. 17 and 20, a tapered expansion pin 165 is provided. The tapered expansion pin has a lower tapered cylindrical body 175 connected to an upper cylindrical body 180. The upper cylindrical body 180 has a channel 182 in a side face 184 and an open channel 183 in a top face 187 of the upper cylindrical body 180. The lower tapered cylindrical body 175 is connected to and coaxial with a bottom face 189 of the upper cylindrical body 180. The tapered expansion pin 165 includes fingers 191 along the bottom face 189, symmetrically located every thirty degrees. Again, the spacing and number of fingers can be varied for a particular purpose. The fingers 191 have tapered tips 193. The device 147 also includes a contraction ring 185, as shown in FIG. 18, that is generally doughnut shaped having a circular passageway 190.

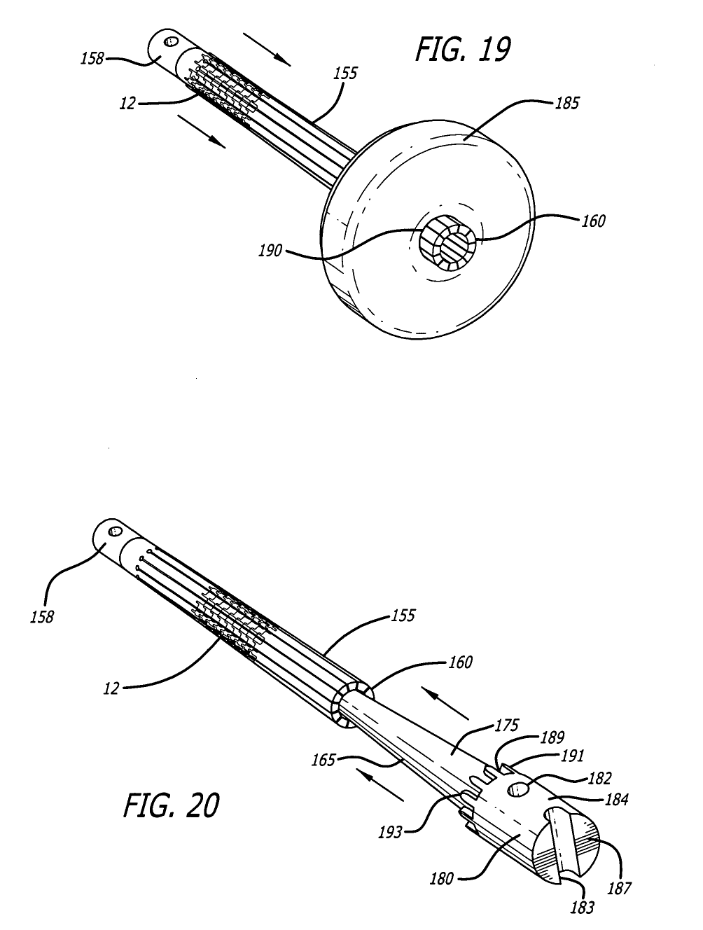

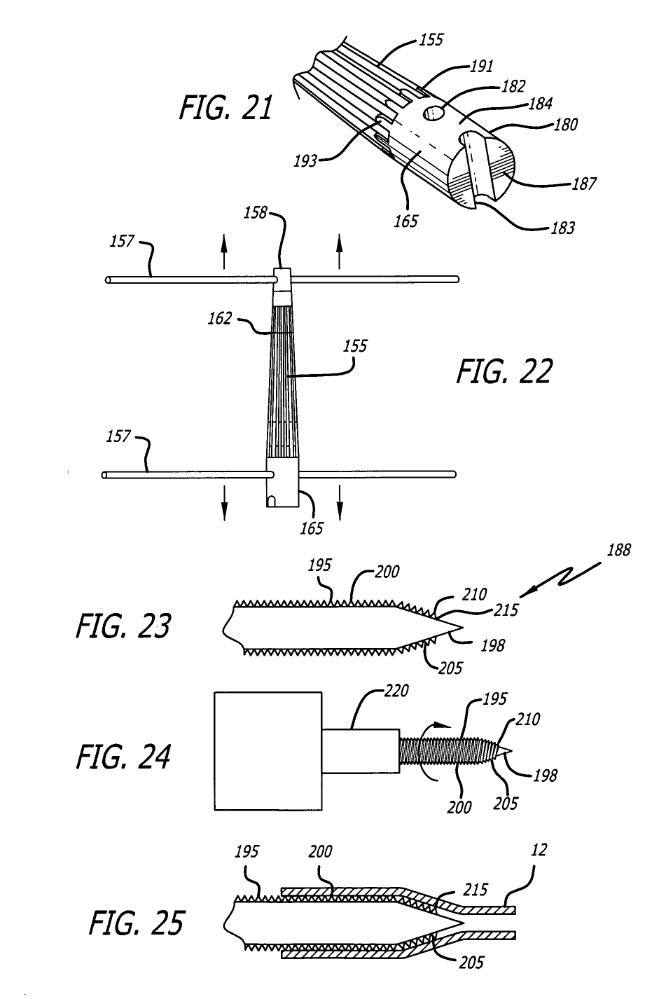

In a first step, the contraction ring 185 is slid over the mandrel 155 commencing at the closed end 158 and until the open end 160. As shown in FIG. 19, a stent 12 that is to become tapered is loaded onto the mandrel 155 at the closed end 158 and is placed between the indicator marks 162. The indicator marks 162 identify the optimal placement of the stent 12 for expansion to a desired shape. After placement of the stent 12 the contraction ring 185 is removed. The tapered pin 165 is inserted into the open end 160 of the mandrel 155 and advanced until the fingers 100 are completely seated in the slots 111, as shown in FIGS. 20 and 21. Thereafter, the stent 12 is in an expanded, tapered configuration. The stent 12 along with the stent expanding device 147 may be heat treated (and cooled) in a manner similar to that described in previous embodiments, and thereafter, the pin 165 may be removed from the mandrel 155 so the expanded stent 12 may be retrieved from the device 147.

As shown in FIG. 22, structures can be employed to facilitate handling of the stent expanding device 147. For example, arms 157 may be slid through the closed channels 182 of the pin 165 and the mandrel 155 to aid in the manipulation of the device 27 and to remove the expansion pin 165 from the expanding mandrel 155. In one instance, the stent expanding device 147 may be automatically moved to and from a chilled solution of isopropyl alcohol. Such an arrangement can be employed with other embodiments previously described, such as the device shown in FIGS. 5 and 6.

In still a further aspect, as shown in FIGS. 23-25, the stent expanding device 188 includes a tapered threaded mandrel 195. The mandrel 195, as shown in FIG. 23, is generally cylindrically shaped with a conical shaped end 198. The mandrel 195 has a shape set portion 200 and an expansion portion 205. The mandrel 195 has threads 210 that commence at a thread commencing point 215 on the expansion portion 205 and continue over the cylindrical body portion 200. The device 188 further includes a motorized rotating member 220, as shown in FIG. 24. The threaded mandrel 195 is loaded into the motorized rotating member 220 and the stent expansion process may be begun. As the mandrel is rotated 220, the stent 12 is guided onto the mandrel 195 over the threads 210 to expand the diameter of the stent 12, as shown in FIG. 25. The stent 12 along with the mandrel 195 may be heat treated in a manner similar to that described in previous embodiments, and thereafter, the expanded stent 12 may be retrieved from the 195.

In another aspect, a procedure for verifying the outer diameter of the mandrel in its expanded state is provided. First the rounding rod is inserted into the mandrel. Next, two O-rings, configured to fit on the unexpanded mandrel, are slid onto the mandrel and position five to ten mm apart near the center of the mandrel. The rounding rod is then removed and the expansion pins are inserted halfway into the mandrel. The expansion crowns are then slid down into the slots of the mandrel. The pins are thereafter pushed completely into the mandrel until they flush against the crowns. Then, a laser micrometer is used to take three measurements of the outer diameter of the expanded mandrel between the O-rings. Each measurement is taken at a one third rotations apart.

The invention described herein has the benefit of improving the efficiency and effectiveness of the stent expansion processes. The invention also lends itself to increased automation or semi-automation thus reducing or eliminating the impact of operator technique on quality. Furthermore, the invention has the advantage of being capable of expanding long stents, up to at least 150 millimeters, and accommodating the expansion of multiple stents simultaneously.

While the specification describes particular embodiments of the present invention, those of ordinary skill can devise variations of the present invention without departing from the inventive concept. Accordingly, it is not intended that the invention be limited, except as by the appended claims.

We Claim:

1. A stent expanding device for expanding the diameter of a stent comprising:
an expansion mandrel having a side and two open ends and at least one longitudinal slot along the side; and
an expansion pin having a cylindrical body and a tapered end.

2. A stent expanding device of claim 1, wherein the at least one longitudinal slot is a plurality of longitudinal slots.

3. The stent expanding device of claim 1, further comprising an expansion crown having teeth configured to engage the slots, and a bore.

4. The stent expanding device of claim 3, wherein the expansion crown is configured to be removably attached to the expansion pin and wherein the teeth are located at substantially evenly spaced intervals on the expansion crown.

5. The stent expanding device of claim 4, wherein the expansion pin further comprises:
an upper cylindrical body having a side face and a top face; and
a closed channel in the side face and an open channel in the top face.

6. The stent expanding device of claim 3, wherein the mandrel includes overlapping members configured to fit into a receiving groove in the mandrel.

7. The stent expanding device of claim 3, further including a rounding rod.

8. The stent expanding device of claim 7, wherein the expansion pin includes a bore extending a length of the expansion pin.

9. The stent expanding device of claim 8, wherein the rounding rod is sized to be received in the bore of the expansion pin.

10. A stent expanding device for expanding a diameter of a stent comprising:
an expansion mandrel having a cavity, a side, two open ends, and a plurality of longitudinal slots along the side, the slots being spaced at regular intervals and having a terminus that is continuous with one of the two open ends of the mandrel, the slots commencing at a keyhole in the side of the mandrel, each consecutive slot terminating at opposite ends of the mandrel;
an expansion pin having a cylindrical body and a tapered end;
an expansion crown having teeth and a bore, the teeth being configured to slide into the termini of the slots, the bore being configured to receive the cylindrical body of the expansion pin; and
a rounding rod configured to be received into the cavity of the mandrel.

11. A stent expanding device for expanding a stent into a tapered stent comprising:
a tapered mandrel having a closed end and an open end, and slots along a side of the tapered mandrel, the slots extending from the closed end of the mandrel and terminating at and continuous with the open end of the mandrel; and
a tapered expansion pin having a tapered cylindrical body.

12. The stent expanding device of claim 11, further comprising teeth on the tapered expansion pin, configured to be received into the termini of the slots.

13. The stent expanding device of claim 12, further comprising a contraction ring having a circular passageway, the passageway configured to receive the tapered mandrel.

14. A stent expanding device, for expanding the diameter of a stent, comprising a threaded mandrel having tapered tip.

15. The stent expanding device of claim 14, further including a rotating device for rotating the threaded mandrel.

16. A method of using a stent expanding device comprising:
inserting an expansion pin into an expansion crown to form a loaded expansion pin;
sliding a stent onto a mandrel to form a loaded mandrel;
partially inserting a loaded expansion pin into each end of the mandrel;
sliding expansion crowns down the expansion pins until fingers of the crowns are seated in slots of the mandrel; and
completely inserting the loaded expansion pin into each end of the mandrel to form an expanded stent.

17. A method of using a stent expanding device comprising:
loading one or more stents onto a mandrel having a cut along the length of a side and a cavity; and
inserting an expansion pin into the cavity of the mandrel.

18. A method of expanding a stent comprising:
rotating a threaded mandrel having a tapered tip and feeding one or more stents onto the mandrel while the mandrel rotates.

19. A method of expanding a stent comprising:
sliding a contraction ring over a mandrel with a closed end and an open end;
loading one or more stents onto the mandrel from the closed end;
sliding the contraction ring away from the stents; and
inserting a tapered pin into the open end of the mandrel.
